(19) United States
(12) Patent Application Publication
(10) Pub. No.: US 2018/0364222 A1
(43) Pub. Date: Dec. 20, 2018

(54) Janus particle, tetrahedral structure including Janus particles, method of fabricating Janus particles, and method of detecting biomolecules

(71) Applicant: NATIONAL CHENG KUNG UNIVERSITY, Tainan City (TW)

(72) Inventors: Chang-Shu Kuo, Tainan City (TW); Yu-Hung Lai, Tainan City (TW)

(21) Appl. No.: 15/623,395

(22) Filed: Jun. 15, 2017

(51) Int. Cl. G01N 33/543; G01N 33/542; G01N 33/552; G01N 33/533; G01N 33/569; B01J 13/04

(52) U.S. Cl. CPC G01N 33/54346; G01N 33/542; G01N 33/552; B01J 13/04; G01N 33/533; G01N 33/56983; G01N 33/54306; G01N 33/54326

(57) ABSTRACT

A Janus particle includes a low-dimensional substrate and biomolecules. A surface of the low-dimensional substrate includes a biomolecule-modified region, wherein the biomolecules are attached on the surface of the low-dimensional substrate and in the biomolecule-modified region. The relationship between the surface area of the biomolecule-modified region and the total surface area of the low-dimensional substrate is represented as follows:

(⅕)AS≤AB≤(½)AS
where AB represents the surface area of the biomolecule-modified region, and AS represents the total surface area of the low-dimensional substrate.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates generally to Janus particles and a method of fabricating the same, and more particularly to Janus particles served to detect microorganisms and a method of fabricating the same.

### Description of Related Arts

Recently, the spread and the mutation rate of animal and/or plant virus have become increasingly rapid due to more and more people traveling around the world. Take Asia-pacific region as an example, diseases caused by adenovirus, influenza virus, Zika virus, dengue virus, and so forth, usually break out in this region during a specific season. Therefore, there is always a need to develop methods for detecting and diagnosing virus in a sensitive, rapid, quantitative, and cost-efficient way.

Conventional methods for detecting viruses may be roughly classified into two categories: immunoassay techniques and molecular biology techniques. For the immunoassay technique, enzyme-linked immunosorbent assay (ELISA) and gold immunochromatography assay (GICA) are often applied for detecting virus. In detail, ELISA is a high-sensitive semi-quantitative assay that is often carried out in a simple way. Thus, ELISA is often used to detect numerous specimens in small quantity. However, ELISA is a time-consuming assay since repeat rinse processes often need to be conducted during the whole process for detecting virus. In addition, the advantage of GICA is that it is a time-efficient, low-cost, and simple method. However, since the sensitivity of GICA is not high enough, it is almost impossible to measure the amount of the viruses in a specimen by using GICA. For the molecular biology technique, it often uses polymerase chain reaction (PCR) for detecting viruses. It is advantageous because of its high sensitivity and high specificity. However, PCR requires expensive equipment and skilled technicians, and it is also time-consuming because the replication of a specific segment of DNA takes time. Furthermore, rapid diagnostic tests for virus usually take at least 30 minutes before the result comes out, and the accuracy of the result is only about 60-70%, which means that medical professionals may not make a decision solely based on the diagnostic result. Therefore, the medical professionals require other diagnostic results to decide whether a specific type of viruses exists or not. In sum, there is still a need to develop a new detecting method without the drawbacks in the conventional detecting methods, such as low-accuracy and time-consuming.

### SUMMARY OF THE PRESENT INVENTION

In light of the above, Janus particles used to detect microorganisms and a method of fabricating the same are disclosed in accordance with the embodiments of the present invention and may successfully overcome the technical drawbacks in the convention technique.

According to one embodiment of the invention, a Janus particle is disclosed and includes a low-dimensional substrate and biomolecules. The surface of the low-dimensional substrate includes a biomolecule-modified region, and the biomolecules are attached on the surface of the low-dimensional substrate and in the biomolecule-modified region. The relationship between the surface area of the biomolecule-modified region and the total surface area of the low-dimensional substrate is represented as follows: (⅕)AS≤AB≤(½)AS, where AB represents the surface area of the biomolecule-modified region and AS represents the total surface area of the low-dimensional substrate.

According to another embodiment of the present invention, a tetrahedral structure is disclosed and includes a microorganism and four Janus particles. The microorganism includes a surface and biomolecules disposed on the surface. The Janus particles surround the microorganism, and each of the Janus particles includes low-dimensional substrate and other biomolecules. The surface of the low-dimensional substrate has a biomolecule-modified region. The biomolecules of the Janus particles are disposed in the biomolecule-modified region. Two ends of each of the biomolecules of the Janus particles are respectively attached to the surface of the low-dimensional substrate and each of the biomolecules of the microorganism.

According to still another embodiment of the present invention, a method for fabricating the Janus particle is disclosed and includes the following steps: providing at least a low-dimensional substrate; adsorbing the low-dimensional substrate on a surface of a fibrous web structure; performing a heating process until portions of the low-dimensional substrate are submerged into the fibrous web structure to constitute a submerged portions, and other portions of the low-dimensional substrate are exposed from the surface of the fibrous web structure to constitute a protruding portions, where the ratio of the surface area of the protruding portions to the surface area of the low-dimensional substrate is between 0.2-0.5; forming a surface modification layer on the surface of the protruding portions; detaching the low-dimensional substrate from the surface of the fibrous web structure after the step of forming the surface modification layer; and disposing a biomolecule layer on the surface modification layer, where the biomolecule layer is attached to the surface modification layer.

According to yet another embodiment of the present invention, a method of detecting biomolecules is disclosed and includes the following steps: providing Janus particles, wherein each of which includes a low-dimensional substrate and biomolecules, wherein the surface of the low-dimensional substrate includes a biomolecule-modified region, wherein the biomolecules respectively include a fixed end and a free end, wherein each of the fixed ends is attached to the surface of the low-dimensional substrate in the biomolecule-modified region, wherein the relationship between the surface area of the biomolecule-modified region and the surface area of the low-dimensional substrate is represented as follows (⅕)AS≤AB≤(½)AS, where AB represents a total surface area of the biomolecule-modified region and AS represents a total surface area of the low-dimensional substrate; providing a microorganism including further biomolecules disposed on the surface of the microorganism; and mixing the Janus particles and the microorganism so that the free end of each of the biomolecules on the surface of the Janus particles is bound to each of the biomolecules disposed on the surface of the microorganism.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

For more complete understanding of the embodiments of the present invention and their advantage, reference is now made to the following description, taken in conjunction with accompanying drawings, in which:

FIG. 1 is a schematic diagram of a Janus particle in accordance with one embodiment of the present invention.

FIG. 2 is a schematic diagram of a tetrahedral structure in accordance with one embodiment of the present invention.

FIG. 3 is a schematic diagram of low-dimensional substrates adsorbed on the surface of a fibrous web structure in accordance with one embodiment of the present invention.

FIG. 4 is a schematic diagram of a solution containing samples and Janus particles in accordance with one embodiment of the present invention.

FIG. 5 is an electron microscope image of a self-assembly tetrahedral structure in accordance with one embodiment of the present invention.

FIG. 6 is a diagram of the diameters of particles versus the ratios of the number of Janus particles to the number of Hepatitis B virus (HBV) in accordance with one embodiment of the present invention.

FIG. 7 is a particle size distribution graph of a solution containing self-assembly tetrahedral structures and excess Janus particles in accordance with one embodiment of the present invention.

FIG. 8 is a schematic diagram of FRET intensity versus wavelength at various concentrations of surface-modified polymer beads in accordance with one embodiment of the present invention.

FIG. 9 is a schematic diagram of FRET intensity versus wavelength with or without magnets to gather magnetic tetrahedral structures in accordance with one embodiment of the present invention.

### DETAILED DESCRIPTION OF THE PREFERRED EMBODIMENT

The invention will be described more fully hereinafter with reference to the accompanying drawings, in which example embodiments of the invention are shown. This invention may, however, be embodied in many different forms and should not be construed as limited to the example embodiments set forth herein. Rather, the disclosed embodiments are provided so that this disclosure will be thorough and complete, and will fully convey the scope of the invention to those skilled in the art. In the drawings, the size and relative sizes of components and regions may be exaggerated for clarity unless express so defined herein.

The terminology used herein is for describing particular embodiments only and is not intended to be limiting. As used herein, the singular terms “a”, “an,” and “the” may be intended to include the plural forms as well, unless the context clearly indicates otherwise. The terms “comprises,” “comprising,” “includes” and/or “including” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the following paragraphs, Janus particles, tetrahedral structures containing Janus particles, methods for fabricating Janus particles, and methods for detecting biomolecules using Janus particles are disclosed in detail. Processes and steps applied in the embodiments below are, except otherwise specified, routine processes and steps. Besides, the materials and reagents used in the embodiments below may be obtained from Sigma-Aldrich or other suitable chemical suppliers.

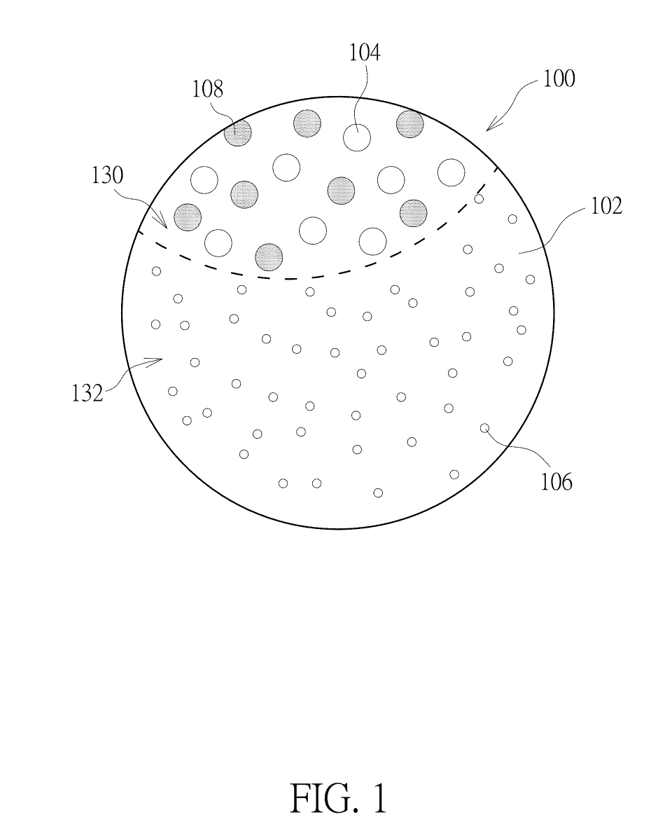

FIG. 1 is a schematic diagram of a Janus particle in accordance with one embodiment of the present invention. A Janus particle 100 includes a low-dimensional substrate 102 and biomolecules 104. The surface of the low-dimensional substrate 102 includes a biomolecule-modified region 130. The biomolecules 104 are attached on the surface of the low-dimensional substrate 102 and in the biomolecule-modified region 130. The relationship between the surface area of the biomolecule-modified region 130 and the total surface area of the low-dimensional substrate 102 is represented as follows: (⅕)AS≤AB≤(½)AS, preferably (¼)AS≤AB≤(⅓)AS, where AB represents the surface area of the biomolecule-modified region and AS represents the total surface area of the low-dimensional substrate.

It should be noted that the term “Janus particle” disclosed throughout the specification should be interpreted as a particle whose surface has at least two distinct chemical and/or physical properties. For instance, one side of the Janus particle 100 may have high specificity to a specific antigen, magnetic properties, the ability to emit fluorescence, and/or the ability to provide fluorescence resonance energy transfer (FRET) property. The other side of the particle, by contrast, may not have magnetic properties, the specificity to a specific antigen, and/or fluorescence ability.

The low-dimensional substrate 102 above may refer to a substrate whose dimension along every orientation is less than 1000 nanometers (nm), preferably less than 500 nm. The low-dimensional substrate 102 may be a spherical substrate, a pillar-shaped substrate, or a dumbbell-shaped substrate, but not limited thereto, and may be made of ceramic or biocompatible materials. According to one embodiment of the present invention, the low-dimensional substrate 102 is a SiO2 spherical substrate with a diameter of about 10-1000 nm, preferably 500 nm. In addition, based on different requirements, the surface and/or the body of the low-dimensional substrate 102 may have porous structures. For example, the low-dimensional substrate 102 may be a mesoporous SiO2 spherical substrate with a diameter of about 2-50 nm.

The biomolecules 104 are selected from the group consisting of proteins, peptides, amino acids, nucleic acids, or other suitable biomolecules. Preferably, the biomolecules 104 are avidins or antibodies with the specificity to certain antigens. One end of the biomolecules 104 may be bound to the surface of the low-dimensional substrate 102 by chemical bonds. For a case where the biomolecules 104 are avidins, the surface of the low-dimensional substrate 102 is preferably modified with carboxyl groups (—COOH), which may react with the amino groups (—NH2) in the avidins and generate amide bonds. In addition, the surface of the low-dimensional substrate 102 may be modified with epoxide groups, which may react with the amino groups (—NH2) in the avidins and generate carbon-nitrogen bonds (C—N). It should be noted that the types of chemical bonds disclosed above are for illustration purposes only and there may be other ways to bind the biomolecules to the surface of the low-dimensional substrate 102.

The biomolecule-modified region 130 is a continuous region distributed on one side of the low-dimensional substrate 102. In other words, the biomolecule-modified region 130 does not occupy the entire surface of the low-dimensional substrate 102. For a case where the biomolecules 130 are disposed only in the biomolecule-modified region 130, the rest of the surface not modified with the biomolecule-modified region 130 may be called a “non-biomolecule-modified region.”

According to one embodiment of the present invention, the surface of the Janus particle 100 may further include a non-biomolecule-modified region 132, which is a continuous region on another side of the Janus particle 100. For example, the non-biomolecule-modified region 132 and the biomolecule-modified region 130 may be respectively on opposite sides of the Janus particle 100 so that the two regions may not overlap each other. Preferably, the non-biomolecule-modified region 132 and the biomolecule-modified region 130 share a common boundary, and the relationship between the low-dimensional substrate (AS), the non-biomolecule-modified region (AM), and the biomolecule-modified region (AB) may be expresses as: AM=AS−AB. Furthermore, certain materials, such as magnetic materials 106, may be disposed in the non-biomolecule-modified region 132, but not limited thereto. In particular, the magnetic materials 106 are preferably magnetic materials with magnetic dipoles, such as paramagnetic materials or ferromagnetic materials, which may show certain magnetic properties in magnetic fields. In addition, for a Janus particle 100 with porous structures, the magnetic materials 106 are not only disposed on the surface of the Janus particle 100, but also disposed in the pores, preferably on the sidewalls of the pores, inside the Janus particle 100.

According to one embodiment of the present invention, the Janus particle 100 may further contain fluorescent molecules 108 attached to the surface of the low-dimensional substrate 102 in the biomolecule-modified region 130. The fluorescent molecules 108 may be FRET donors, FRET acceptors, or other fluorescent molecules.

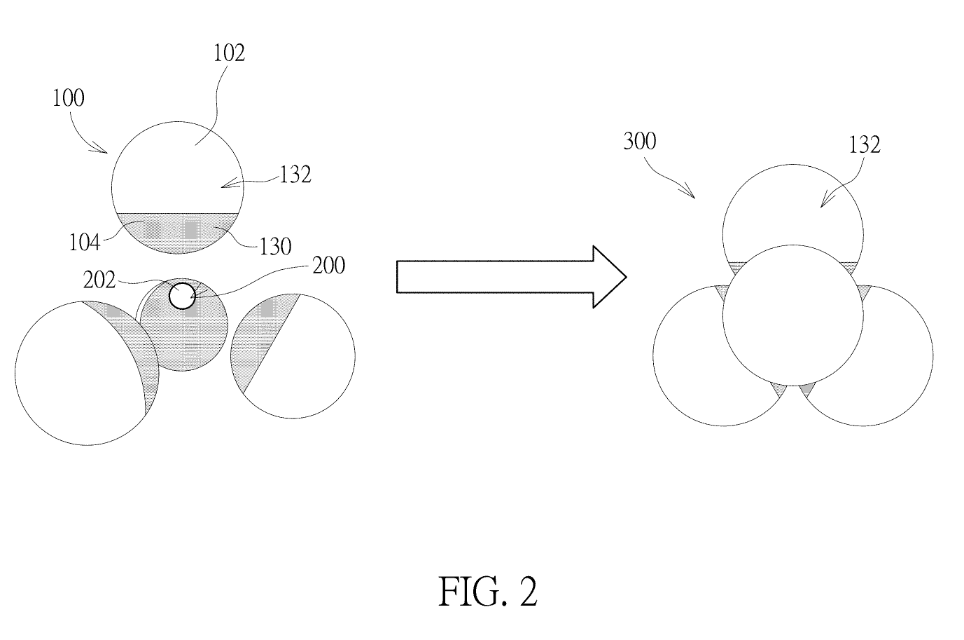

FIG. 2 is a schematic diagram of a tetrahedral structure in accordance with one embodiment of the present invention. As shown in FIG. 2, the Janus particles 100 have structures similar to the structure of the Janus particle shown in FIG. 1. Each tetrahedral structure 300 may contain one microorganism 200 and four Janus particles 100 surrounding the microorganism 200. In particular, the microorganism 200 is in a tetrahedral hole defined by the four Janus particles 100, wherein Janus particles 100 may respectively constitute the apexes of the tetrahedral structure 300. Preferably, every two adjacent Janus particles 100 may be spaced apart from one another at approximately the same distance, but not limited thereto. The microorganism 200 may contain several biomolecules 202 on its surface. One end of each of the biomolecules 104 of the Janus particles 100 may be attached to the surface of the low-dimensional substrate 102, while the other end of each of the biomolecules 104 of the Janus particles 100 may be bound to the biomolecules 202 on the surface of the microorganism 200. Preferably, the relationship between the maximum dimension of the low-dimensional substrate (D), which is taken along a single orientation, and the maximum dimension of the microorganism (VD), which is taken along an single orientation, is represented as: 0.1≤(VD/D)≤0.35, and preferably 0.15≤(VD/D)≤0.3. It should be noted that the technical term “microorganism” disclosed throughout the disclosure refers to an organism with a maximum size of 20-150 nm along a single orientation, having genetic materials, and having the ability to self-replicate, such as virus, but not limited thereto.

For a case where the Janus particles 100 contain fluorescent molecules 108, the surface of the microorganism 200 may be attached with the corresponding fluorescent molecules. For example, when the fluorescent molecule 108 attached on the surface of the Janus particles 100 is one member of the FRET donor-acceptor pair, i.e. a FRET donor or a FRET acceptor, the fluorescent molecule 108 attached on the surface of the microorganism 200 is the other member of the FRET donor-acceptor pair.

In addition, the Janus particles 100 and the microorganism 200 may constitute octahedral structures. For example, six Janus particles 100 and one microorganism 200 may constitute one octahedral structure. The six Janus particles 100 may define an octahedral hole at the center of the octahedral structure, wherein the octahedral hole may be occupied by a single microorganism 200.

The Janus particles 100 and the tetrahedral structure 300 made of Janus particles 100 are disclosed in the embodiments above. Methods for fabricating the Janus particles are disclosed in detail accompanied with FIG. 1 in the following paragraphs.

According to one embodiment of the present invention, a method for fabricating Janus particles is disclosed. First, a low-dimensional substrate is provided and may be made of ceramic materials or biocompatible materials. For example, the low-dimensional substrate may be a SiO2 spherical substrate with a diameter of about 10-1000 nm, preferably 500 nm.

Then, porous structures may be optionally fabricated in the low-dimensional substrate. For example, for the spherical substrate made of SiO¬2, a protection layer, such as a layer made of polyvinylpyrrolidone (PVP), may be formed on the surface of the spherical substrate. Afterwards, the spherical substrate not covered by the protection layer is etched by suitable etchants until pores (also called porous structures) are fabricated in the spherical substrate. Preferably, a mesoporous spherical substrate may be obtained when the etching process is completed.

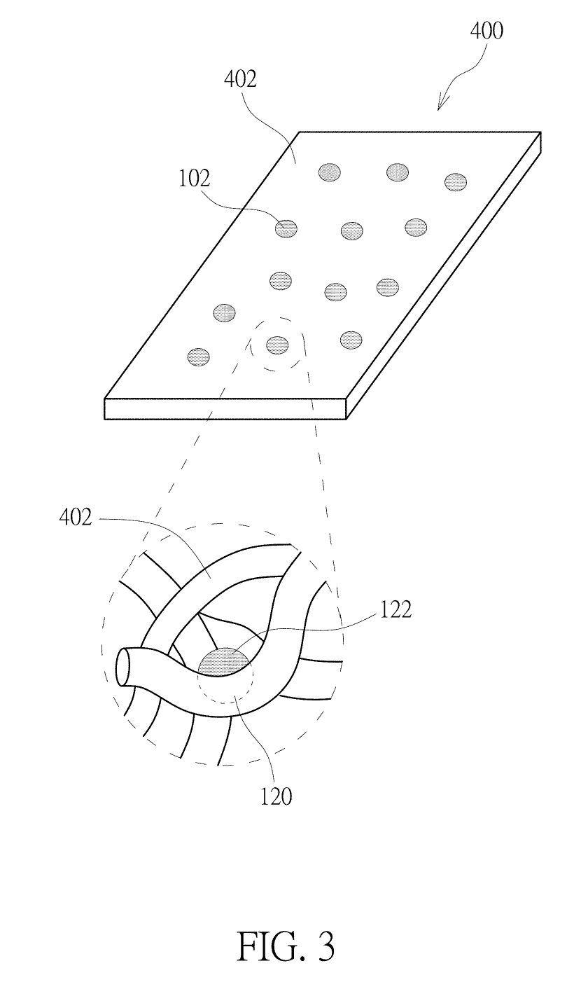

Please refer to FIG. 3. FIG. 3 is a schematic diagram of low-dimensional substrates adsorbed on the surface of a fibrous web structure in accordance with one embodiment of the present invention. Then, the low-dimensional substrates 102 are adsorbed onto the surface of the fibrous web structure 400 consisting of electrospun fibers 402 stacking over one another. The electrospun fibers 402 may be made of single or multiple polymers, wherein each polymer may be a homopolymer or a copolymer selected from the group consisting of acrylic-based polymer, vinyl-based polymer, polyester, and polyamide, but not limited thereto. Preferably, the electrospun fibers 402 is made of two types of polymers, that is, polymethylmethacrylate (PMMA) and poly(4-vinylpyridine) (P4VP), but not limited thereto.

Then, still referring to FIG. 3, a heating process is conducted to have portions of each low-dimensional substrate 102 submerged into the fibrous web structure 400 (i.e. submerged portion 120), and have the other portions of each low-dimensional substrate 102 protrude from the surface of the fibrous web structure 400 (i.e. protruding portion 122). The ratio of the surface area of the protruding portion 122 to the surface area of the low-dimensional substrate 102 is preferably between 0.2-0.5. In other words, the area of the protruding portion 122 is less than that of the submerged portion 120.

Then, a deposition process, such as a chemical vapor deposition process or an aqueous chemical reaction, is conducted to form a surface modification layer on the protruding portion 122 of each low-dimensional substrate 102. In this fabrication stage, the submerged portion 120 is still embedded in the electrospun fibers 402 and thus is not covered by the surface modification layer. According to one embodiment of the invention, the surface modification layer is made of organic molecules that may be bound to the surface of the low-dimensional substrate 102 by one end of each organic molecule. The other end of each organic molecule may contain a certain functional group, such as amino group, which may be bound to biomolecules 104 fabricated in the subsequent process. The organic molecules of the surface modification layer may be (3-aminopropyl)trimethoxysilane (APS), but not limited thereto.

Then, another deposition process, such as gas deposition process, is conducted to form a protection layer, such as paraffin wax, covering the surface modification layer. In this fabrication stage, the submerged portion 120 of each low-dimensional substrate 102 is still embedded in the electrospun fibers 402 and thus is not covered by the protection layer.

Subsequently, the fibrous web structure 402 is removed using organic solvent or other proper methods to detach the low-dimensional substrates 102 from the surface of the fibrous web structure 402. At this time, the surface modification layer and the protection layer may be stacked in sequence on one side of each low-dimensional substrate 102 and may not be stacked on the other side of each low-dimensional substrate 102.

Then, a suitable synthetic method, such as a sol-gel method, for synthesizing magnetic materials 106 on the surface and/or in the pores of the low-dimensional substrate 102 may be conducted in an aqueous solution. The magnetic materials 106 may be ferromagnetic materials or ferromagnetic materials, preferably nanoparticles of Fe2O3 or Fe3O4. During the synthetic process, since the low-dimensional substrates 102 are partly covered with the protection layer, the magnetic materials 106 are restricted to be synthesized in the uncovered region. Afterwards, certain solvent, such as hexane, are applied to remove the paraffin wax on the low-dimensional substrate 102 until the surface modification layer originally underneath the paraffin wax is exposed.

Then, optional fluorescent molecules 108 are attached to the low-dimensional substrate 102 in the biomolecule-modified region 130. The fluorescent molecules 108 may be a certain type of fluorescent molecules, such as FRET donors or FRET acceptors. According to one embodiment of the invention, the fluorescent molecules 108 are FRET donor dyes, such as Marina Blue dyes.

Afterwards, biomolecules 104 are attached to the low-dimensional substrate 102 in the biomolecule-modified region 130 to therefore obtain immunoactive magnetic submicron Janus particles. The types of the biomolecules 104 are similar to those disclosed above and thus the description of which are omitted for the sake of brevity.

It should be noted that, although the processes for attaching the fluorescent molecules 108 and the biomolecules 104 to the low-dimensional substrate 102 are conducted in sequence, the processes may be conducted in reverse order in accordance with other embodiments of the invention.

The methods for fabricating the Janus particles are disclosed in the embodiments above. In the following paragraphs, methods for detecting biomolecules (or viruses) by using the Janus particles are further disclosed. It should be noted that the Janus particles disclosed in the following embodiments may be the Janus particles disclosed in the embodiments above. Therefore, the description of the detailed structures and parts of the Janus particles is omitted for the sake of brevity.

Analogous to the Janus particle disclosed in the embodiment of FIG. 1, each Janus particle 100 of the present embodiment also includes a low-dimensional substrate 102 and biomolecules 104. The surface of the low-dimensional substrate 102 includes a biomolecule-modified region 130. The biomolecules 104 respectively include a fixed end and a free end, wherein each of the fixed ends is attached to the surface of the low-dimensional substrate 102 in the biomolecule-modified region 130. The relationship between the surface area of the biomolecule-modified region (AB) and the surface area of the low-dimensional substrate (AS) is represented as follows (⅕)AS≤AB≤(½)AS, preferably (¼)AS≤AB≤(⅓)AS.

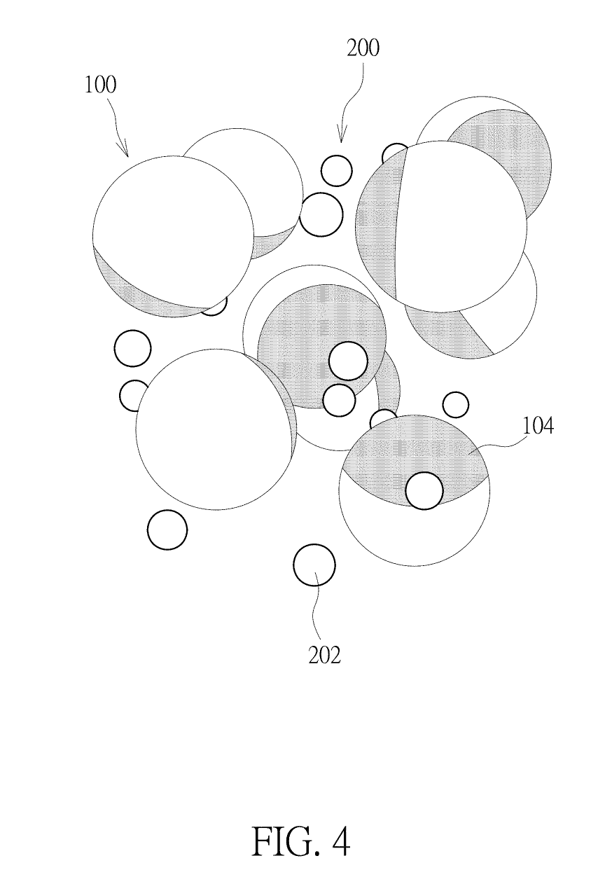

FIG. 4 is a schematic diagram of a solution containing samples and Janus particles in accordance with one embodiment of the present invention. Then, samples, such as microorganisms 200, are added to a solution containing the Janus particles 100. Since the biomolecules 202 on the surface of the microorganisms 200 have the specificity to the free ends of the biomolecules 104 on the surface of the Janus particles 100, each biomolecules 202 is able to be bound to the free end of each biomolecule 104 on the surface of the Janus particles 100 once the microorganisms 200 are mixed with the Janus particles 100. Besides, when each fluorescent molecule 108 attached on the surface of the Janus particles 100 is one member of the FRET donor-acceptor pair, i.e. a FRET donor or a FRET acceptor, each fluorescent molecule attached on the surface of the microorganism 200 is the other member of the FRET donor-acceptor pair.

It should be noted that the relationship between the surface area of the biomolecule-modified region (AB) and the surface area of the low-dimensional substrate (AS) is represented as: (⅕)AS≤AB≤(½)AS, wherein the relationship between the maximum dimension of the low-dimensional substrate (D), which is taken along a single orientation, and the maximum dimension of the microorganism (VD), which is taken along an single orientation, is represented as: preferably 0.15≤(VD/D)≤0.3. Thus, the Janus particles 100 with a certain diameter may capture the microorganisms 200 with a certain diameter to therefore constitute tetrahedral structures as shown in FIG. 2. In particular, single microorganism is in a tetrahedral hole defined by the four Janus particles. By analyzing the number of the tetrahedral structure by certain apparatus, such as dynamic light scattering (DLS) apparatus, the number of the microorganisms can also be determined.

To assure that all the microorganisms 200 is captured and surrounded by the Janus particles 100, the microorganisms 200 should be slowly added to the solution containing the Janus particles 100, and the ratio of the number of the Janus particles 100 to the number of the microorganisms 200 should be much greater than 10:1, preferably from 30:1 to 80:1.

It should be noted that, for a case where the relationship between the surface area of the biomolecule-modified region (AB) and the surface area of the low-dimensional substrate (AS) is represented as: (½)AS<AB, the tetrahedral structures may not be produced successfully even if the Janus particles are mixed with the microorganisms. The reason is that the surface area of the biomolecule-modified region in this case is large enough to simultaneously bind to two or more microorganisms. Accordingly, the Janus particles are not able to capture the microorganisms with a certain particle size, and the number of the microorganisms cannot be determined based on the number of the tetrahedral structures.

In addition, in a case where magnetic materials and/or fluorescent molecules constitute parts of the Janus particles, and the surface of the microorganisms is modified with fluorescent molecules, the number of the tetrahedral structures as well as that of the microorganisms may be determined by FRET signal strength. Besides, the number of the microorganism may also be determined using centrifugation or precipitation to collect the tetrahedral structures. Additionally, FRET signal strength may be further boosted by gathering magnetic tetrahedral structures in a certain region through applying a magnetic field to the solution containing the magnetic tetrahedral structures.

In order to enable those of ordinary skill in the art to make and use the present invention, several examples, such as Preparation Examples, Production Examples, and Test Examples are disclosed in the following paragraphs in detail. It should be noted that the examples disclosed below are for illustration purposes only and should not be construed as limiting the inventive concept to any particular example. In other words, the types, quantities, and ratios of the materials, the processes, and so forth disclosed in the examples may be properly modified, without departing from the scope of the prevent invention, and still regarded as embodiments of the present invention.

### Preparation of Janus Particles

### Preparation Example 1—Preparation of Janus Particles with Immunoactivity and Fluorescent Molecules

First, SiO2 spherical particles with a diameter of 500 nm are provided. The spherical particles are then cleaned up and treated with polyvinylpyrrolidone (PVP) (Sigma-Aldrich, average M.W.=10,000) to form a layer of PVP on the surface of the spherical particles. The coated spherical substrates are then treated with aqueous sodium hydroxide to produce mesoporous submicron SiO2 spherical substrates with a diameter of 500 nm. Afterwards, the mesoporous SiO2 spherical particles are adsorbed onto the surface of a fibrous structure. The fibrous structure may contain at least one electrospun fiber made of polymethylmethacrylate (PMMA) (Sigma-Aldrich, average M.W.=120,000) and poly(4-vinylpyridine) (P4VP) (Sigma-Aldrich, average M.W.=60,000). Subsequently, an environmental temperature is raised to 158° C. and kept for a period of time to have the mesoporous SiO2 spherical particles partly submerged into the electrospun fibers. Preferably, two-thirds of the total surface of each particle may be embedded in the electrospun fibers, while one-third of the total surface of each particle may be exposed from the electrospun fiber. In detail, the surface energy of the electrospun fibers and that of the spherical particles are under equilibrium conditions at a steady temperature, which causes certain amounts of the spherical particles are submerged in the electrospun fibers. Additionally, the surface energy of the electrospun fibers may be adjusted by varying their composition, such as the ratio of PMMA to P4VP. By adjusting the surface energy of the electrospun fibers and the environmental temperature, the amounts of the submerged portions of the spherical particles may be well controlled. A chemical vapor deposition process is then conducted to deposit 3-Aminopropyltrimethoxysilane (APS) (Sigma-Aldrich) on the exposed surfaces of the spherical particles and thus form a surface modified with amino groups. As a result, submicron Janus particles are obtained. Afterwards, the electrospun fibers are dissolved in organic solvent. The surface modified with the amino groups are then bound to Marina Blue fluorescent dye (ThermoFisher), and IgG anti-biotin antibodies are also bound to the surface modified with the aid of amino groups through N-(3-Dimethylaminopropyl)-N′-ethylcarbodiimide hydrochloride (EDC) (Sigma-Aldrich). The fabricated immunoactive Janus particles are abbreviated as A-1.

### Preparation Example 2—Preparation of Janus Particles with Immunoactivity and Fluorescent Molecules

The process carried out in Preparation Example 2 is similar to that carried out in Preparation Example 1. The major characteristic of Preparation Example 2 is that half of the Janus particles are modified with the FRET donors, such as Marina Blue, and the other half of the Janus particles are modified with the FRET acceptors, such as 6-(7-Nitrobenzofurazan-4-ylamino) hexanoic acid. The Janus particles produced in Preparation Example 2 are abbreviated as A-2.

### Preparation Example 3—Preparation of Janus Particles with Immunoactivity, Magnetic Properties, and Fluorescent Molecules

The process carried out in Preparation Example 3 is similar to that carried out in Preparation Example 1. The major characteristic of Preparation Example 3 is that iron oxide is further synthesized after the step of modifying the surface of the spherical particles with amino groups and before the step of attaching the Marina Blue fluorescent dye to the surface-modified spherical particles. The corresponding processes are disclosed as follows. Another vapor depositing process is conducted after the surface of the spherical particles is modified with the amino groups to cover the exposed surface of the spherical particles with ultrapar wax. The electrospun fibers are then dissolved by organic solvent to obtain mesoporous Janus particles where one-third of the spherical surface is covered in the ultrapar wax. Then, the mesoporous Janus particles are dispersed in an aqueous solution, and iron oxide are synthesized in the pores of the mesoporous Janus particles not covered by the ultrapar wax. The ultrapar wax is then dissolved by applying hexane, and submicron magnetic Janus particles are thus fabricated. The Janus particles produced in Preparation Example 3 are abbreviated as A-3.

### Preparation Example 4—Preparation of Janus Particles with Immunoactivity, Magnetic Properties, and Fluorescent Molecules

The process carried out in Preparation Example 4 is similar to that carried out in Preparation Examples 2 and 3. The major characteristic of Preparation Example 4 is that half of the Janus particles are modified with the FRET donors, such as Marina Blue, and the other half of the Janus particles are modified with the FRET acceptors, such as 6-(7-Nitrobenzofurazan-4-ylamino) hexanoic acid. Besides, only the Janus particles modified with the FRET donors (or the FET acceptors) contain iron oxide and the Janus particles modified with the FRET acceptors (or the FRET donors) do not contain iron oxide. The Janus particles produced in Preparation Example 4 are abbreviated as A-4.

### Preparation Example 5—Preparation of Janus Particles with Immunoactivity

The process carried out in Preparation Example 5 is similar to that carried out in Preparation Example 1. The major difference is that the Janus particles fabricated in Preparation Example 5 are not attached with the fluorescent dyes. Thus, the fabricated Janus particles (A-5) do not have FRET characteristics.

### Production of Tetrahedral Structures

### Production Example 1—Use of Hepatitis B Virus (HBV)

The Janus particles (A-1) prepared in Preparation Examples 1 are distributed in a solution. Then, Hepatitis B virus (HBV) with a diameter of 60-70 nm is slowly added to the solution containing the Janus particles (A-1). A co-assembly process may be carried out automatically to generate tetrahedral structures (abbreviated as B-1) once the HBV is mixed with the Janus particles (A-1). It should be noted that, in order to assure that all the HBV is captured and surrounded by the Janus particles (A-1), the ratio of the number of the Janus particles (A-1) to the number of the HBV should be much greater than 4:1, such as 30:1.

### Production Example 2 and 3—Use of Surface-Modified Polymer Beads

The Janus particles (A-1 and A-3) respectively prepared in Preparation Examples 1 and 3 are dispersed in solutions. Then, surface-modified polymer beads with diameters of 60-70 nm are slowly added to the solutions respectively containing the Janus particles (A-1 and A-3). A co-assembly process may be carried out automatically to generate tetrahedral structures (abbreviated as B-2 and B-3) once the surface-modified polymer beads are mixed with the Janus particles (A-1 and A-3)

The surface-modified polymer beads may be surface-carboxylated polyacrylonitrile nanobeads (CH470, commercially-available polymer beads) with diameters of 80 nm. The surface of the surface-modified polymer bead may be bound to biotin serving as antigens through N-(3-Dimethylaminopropyl)-N′-ethylcarbodiimide hydrochloride (EDC). Since the polymer beads CH470 contain fluorescent dyes, i.e. Chromeon 470, the fluorescent dyes, Marina Blue, in the Janus particles (A-1) may serve as FRET donors, and the fluorescent dyes, Chromeon 470, may serve as FRET acceptors. When the distance between the FRET donor and the FRET acceptor is short enough, such as 1-10 nm, the phenomenon of FRET is able to occur.

### Production Example 4—Use of HBV

The Janus particles (A-2) prepared in Preparation Examples 2 are distributed in a solution. Then, HBV with a diameter of 60-70 nm is slowly added to the solution containing the Janus particles (A-2). A co-assembly process may be carried out automatically to generate tetrahedral structures (abbreviated as B-4) once the HBV is mixed with the Janus particles (A-2). It should be noted that, to assure that all the HBV is captured and surrounded by the Janus particles (A-2), the ratio of the number of the Janus particles (A-2) to the number of the HBV should be much greater than 4:1, such as 30:1. Besides, most of the tetrahedral structures (B-4) are made of at least one Janus particle with FRET donor (Marina Blue) and at least one Janus particle with FRET acceptor (6-(7-Nitrobenzofurazan-4-ylamino) hexanoic acid). Preferably, the tetrahedral structure (B-4) is made of two Janus particles with FRET donors and two Janus particles with FRET acceptors.

### Production Example 5—Use of HBV

The Janus particles (A-4) prepared in Preparation Examples 4 are dispersed in a solution. Then, HBV with a diameter of 60-70 nm is slowly added to the solution containing the Janus particles (A-1). A co-assembly process may be carried out automatically to generate tetrahedral structures (abbreviated as B-5) once the HBV is mixed with the Janus particles (A-4). It should be noted that, to assure that all the HBV is captured and surrounded by the Janus particles (A-4), the ratio of the number of the Janus particles (A-4) to the number of the HBV should be much greater than 4:1, such as 30:1. Besides, most of the tetrahedral structures (B-5) are made of at least one Janus particle with FRET donor (Marina Blue) and at least one Janus particle with FRET acceptor (6-(7-Nitrobenzofurazan-4-ylamino) hexanoic acid). Preferably, each of the tetrahedral structures (B-5) is made of two Janus particles with FRET donors and two Janus particles with FRET acceptors.

### Examination of Biomolecules

### Test Example 1—Verification of Tetrahedral Structures Using an Electron Microscope

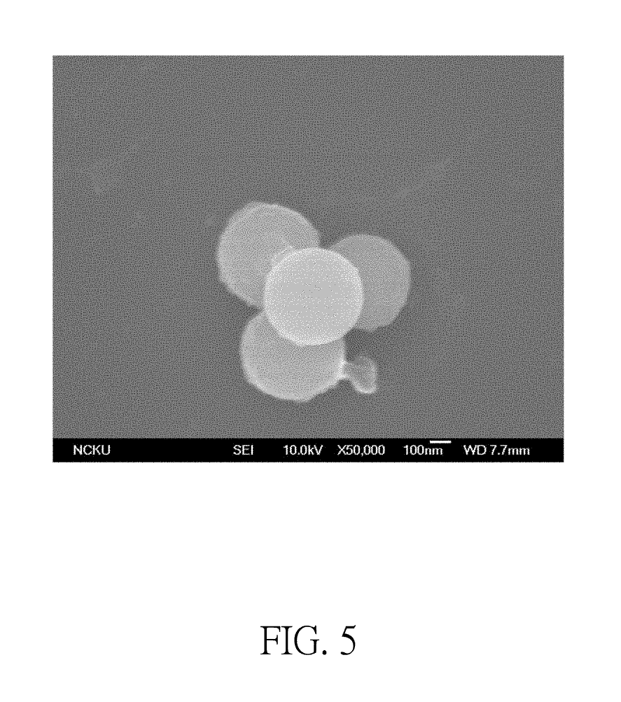

The tetrahedral structures (B-1) produced in Production Example 1 are examined by an electron microscope. FIG. 5 shows that four immunoactive Janus particles and one virus constitute a tetrahedral structure. The space formed among the immunoactive Janus particles has specific dimensions, which allows a virus with a certain diameter (such as diameters of 50-100 nm, preferably 80-100 nm) to be captured. Thus, the accuracy of the method for detecting viruses can be improved. For example, the tetrahedral hole of the tetrahedral structure may accommodate a virus with a diameter of approximately 100 nm.

### Test Example 2—Verification of Tetrahedral Structures Using Dynamic Light Scattering (DLS)

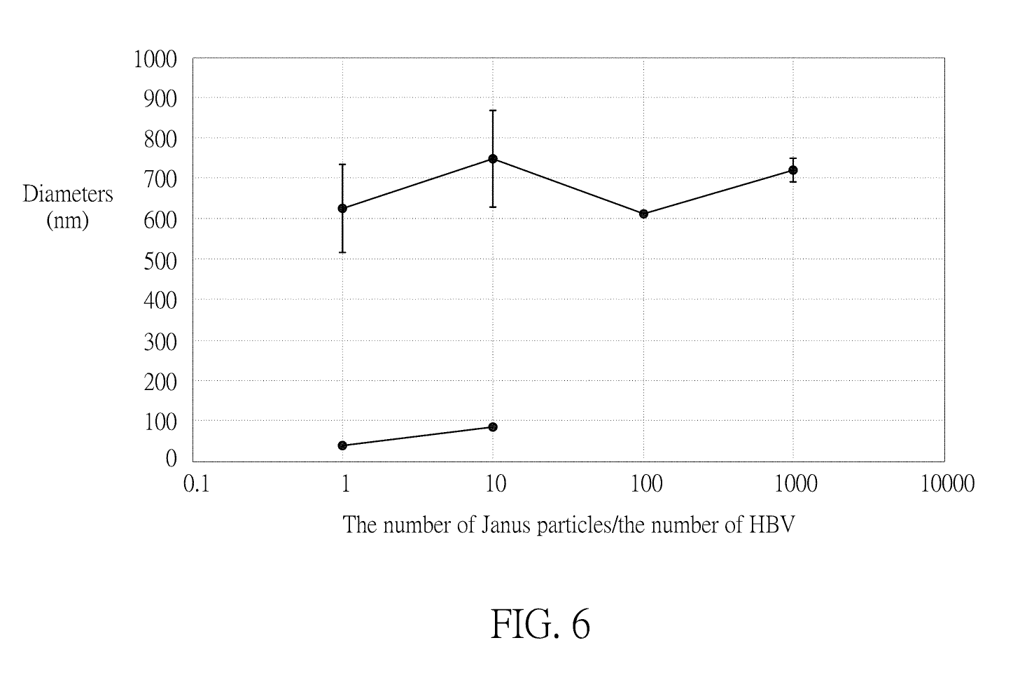

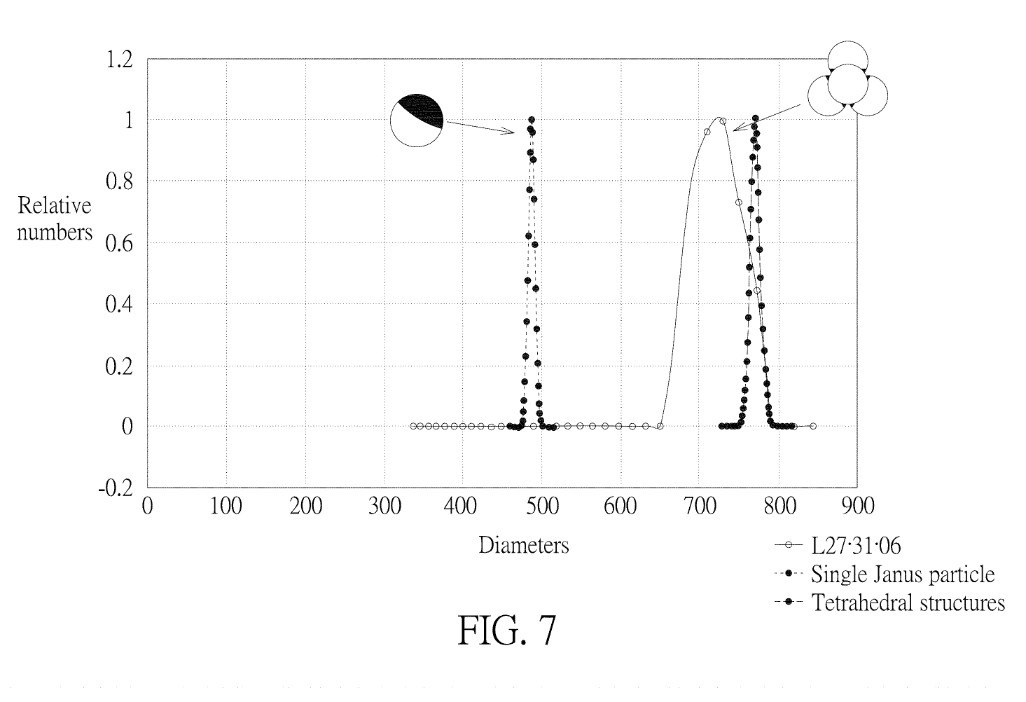

The tetrahedral structures (B-1) produced in Production Example 1 are analyzed using dynamic light scattering (DLS). FIG. 6 is a diagram of the diameters of particles versus the ratio of the number of Janus particles to the number of Hepatitis B virus (HBV) in accordance with one embodiment of the present invention. As shown in FIG. 6, when the ratio of the number of the Janus particles to that of the HBV is greater than 10, the signal corresponding to particles with a size under 100 nm, i.e. HBV, is disappeared, which means that all the HBV is captured by the immunoactive Janus particles. FIG. 7 is a particle size distribution graph of a solution containing self-assembly tetrahedral structures and excess Janus particles in accordance with one embodiment of the present invention. As shown in FIG. 7, a curve labeled with symbols L27-31-06 represents a particle size distribution of a solution containing tetrahedral structures and excess Janus particles. Since the average particle size is decided by the ratio of the tetrahedral structures to the number of the Janus particles in the solution, the number of HBV can be derived by analyzing the distribution of the curve. Besides, the curve on the left-hand side of FIG. 7 represents a particle size distribution of a solution containing only independent Janus particles, while the curve on the right-hand side of FIG. 7 represents a particle size distribution of a solution containing only tetrahedral structures.

### Test Example 3—Verification of Tetrahedral Structures Based on FRET Signal

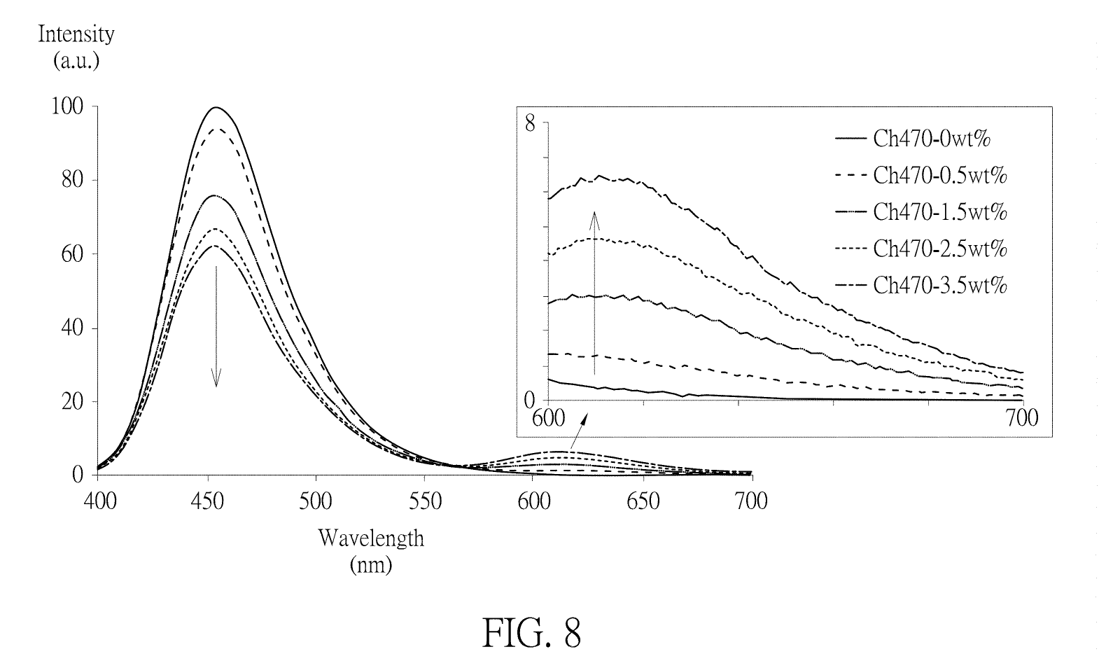

Solutions with the same concentration of Janus particles (A-1) and different concentrations of Chromeon 470 (corresponding to the surface-modified beads in Production Example 2), from 0 wt % to 3.5 wt %, are prepared. The solution may thus have different concentrations of the tetrahedral structures (B-2). Then, the mixed solutions are examined by FRET, and the result is shown in FIG. 8. FIG. 8 is a schematic diagram of FRET intensity versus wavelength at various concentrations of surface-modified polymer beads in accordance with one embodiment of the present invention. When look at the FRET signal at a wavelength of 611 nm, the increase in the signal strength is proportional to the increase in the concentration of Chromeon 470. Accordingly, the number of Chromeon 470, i.e. the number of the surface-modified beads, may be effectively derived based on the changes in FRET signal strength at certain wavelength.

### Test Example 4—Verification of Tetrahedral Structures Based on FRET Signal

The tetrahedral structures (B-4) produced in Production Example 4 is examined, where most of the tetrahedral structure (B-4) are made of one HBV, two Janus particles with FRET donors (Marina Blue), and two Janus particles with FRET acceptors (6-(7-Nitrobenzofurazan-4-ylamino)hexanoic acid). When the distance between two adjacent Janus particles in each tetrahedral structure is less than 10 nm, the corresponding FRET donor-acceptor pairs can generate FRET signals. Therefore, the number of HBV can be derived by measuring the FRET signal strength.

### Test Example 5—Verification of Tetrahedral Structures Based on FRET Signal with Magnetic Fields

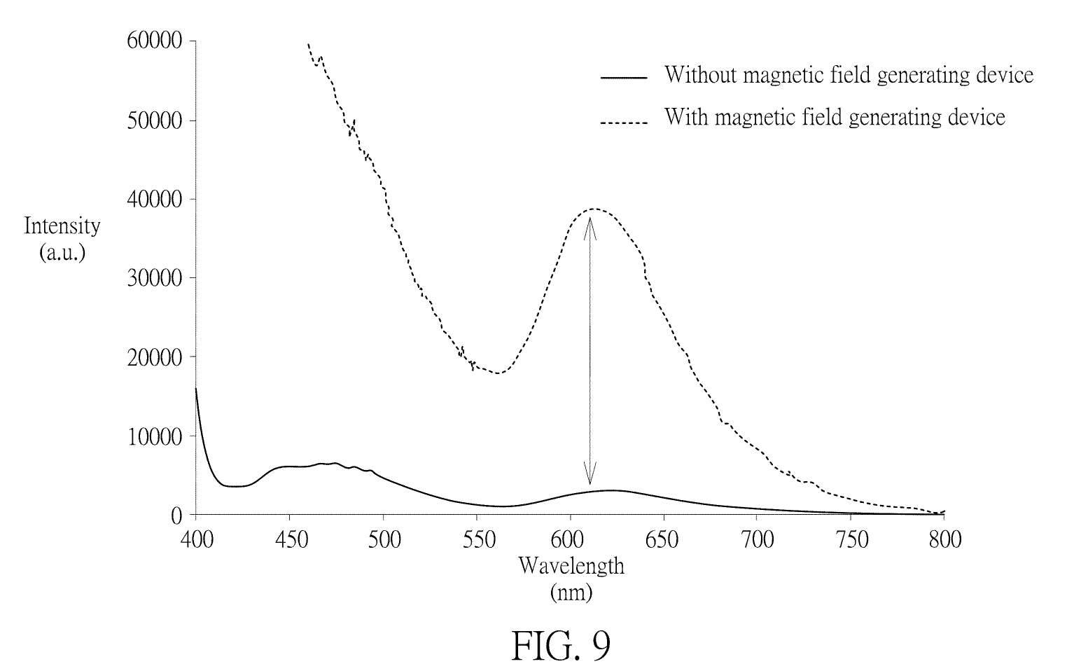

The tetrahedral structures (B-3) produced in Production Example 3 are examined in Test Example 5. The process carried out in Test Example 5 is similar to the process carried out in Test Example 4, the major different between the two Test Examples is that Test Example 5 further uses magnetic fields generating device, such as magnets or electromagnets, to generate magnetic fields. When the magnetic fields are applied to the solution containing the tetrahedral structures (B-3), the tetrahedral structures (B-3) may be attracted by the magnetic fields and thus gathered in a certain region of the solution. For example, the tetrahedral structures (B-3) may be gathered in a place close to the sidewalls of the container. The signal strength of the corresponding FRET can be boosted effectively by concentrating the tetrahedral structures (B-3). As demonstrated in FIG. 9, the signal strength at the wavelength of 611 nm is increased by 13.4 times by applying magnetic fields compared with that without magnetic fields.

It should be noted that, because only one member of the FRET donor-acceptor pair, i.e. donors or acceptors, may be disposed on the surfaces of the Janus particles (A-3), even though the Janus particles (A-3) (also called free magnetic Janus particles) failing to capture any surface-modified polymer beads are also attracted by the applied magnetic field, and the distance between two adjacent free magnetic Janus particles is reduced down to 1-10 nm, no FRET signals (also called noise which may negatively affect the accuracy of the number of the virus) may be given off by the free magnetic Janus particles.

### Test Example 6—Verification of Tetrahedral Structures Based on FRET Signal

The tetrahedral structures (B-5) produced in Production Example 5 are examined in Test Example 6. The process carried out in Test Example 6 is similar to the process carried out in Test Example 4, the major different between the two Test Examples is that Test Example 6 further uses magnetic field generating device, such as magnets or electromagnets, to generate magnetic fields. When the magnetic fields are applied to the solution containing the tetrahedral structures (B-5), the tetrahedral structures (B-5) may be attracted by the magnetic fields and thus gathered in a certain region of the solution. For example, the tetrahedral structures (B-5) may be gathered in a place close to the sidewalls of the container. The corresponding FRET signal can be boosted effectively by 2-3 times through concentrating the tetrahedral structures (B-5).

It should be noted that only the Janus particles (A-4) modified with the FRET donors (or the FET acceptors) contain iron oxide, and the Janus particles (A-4) modified with the FRET acceptors (or the FRET donors) do not contain iron oxide. Thus, the Janus particles that can be attracted by the magnetic fields all have the same type of fluorescent molecules (that is, one type of FRET donors or FRET acceptors). Accordingly, even though the Janus particles (A-4) (also called free magnetic Janus particles) failing to capture any HBV are also attracted by the applied magnetic field and the distance between two adjacent free magnetic Janus particles is reduced to 1-10 nm, no FRET signals (also called noise which may negatively affect the accuracy of the result) may be given off by the free magnetic Janus particles.

### Test Example 7—Separation of Tetrahedral Structures Using Centrifugation

Since the tetrahedral structures (B-1 and B-2 and B-3 and B-4 and B-5) produced in Example 1, 2, 3, 4 and 5 have the density greater than the density of single Janus particle and single virus (or beads), the tetrahedral structures may be concentrated and collected by centrifugation and the number of the viruses or the beads may be further determined.

Compared with conventional methods for detecting viruses, the Janus particles and the co-assembly structures made of the Janus particles disclosed in the embodiments of the present invention can detect and diagnose viruses in a sensitive, rapid, quantitative, and cost-efficient way. In addition, since the tetrahedral structures disclosed in the embodiments above have the density greater than the density of single Janus particle and single virus (or bead), the tetrahedral structures may be concentrated and collected by centrifugation and the number of the virus or the beads may be further determined. Furthermore, the magnetic tetrahedral structures may be rapidly gathered by applying magnetic fields to the solution containing the magnetic tetrahedral structures. By gathering the magnetic tetrahedral structures, the FRET signal can be boosted effectively by several times during the process of detecting FRET signal given off from the FRET donor-acceptor pairs. The accuracy of the result is therefore improved. In sum, the structures and methods disclosed in the embodiments of the present invention can be used to rapidly diagnose and detect viruses in aqueous solution.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A Janus particle, comprising:
a low-dimensional substrate, wherein a surface of said low-dimensional substrate comprises a biomolecule-modified region; and
a plurality of biomolecules attached to said surface of said low-dimensional substrate in said biomolecule-modified region, wherein a relationship between a surface area of said biomolecule-modified region and a surface area of said low-dimensional substrate is represented as follows:
(⅕)AS≤AB≤(½)AS,
wherein
AB represents a total surface area of said biomolecule-modified region and
AS represents a total surface area of said low-dimensional substrate.

2. The Janus particle, as recited in claim 1, wherein said low-dimensional substrate is a spherical substrate, a pillar-shaped substrate, or a dumbbell-shaped substrate.

3. The Janus particle, as recited in claim 1, wherein said low-dimensional substrate is a mesoporous spherical substrate.

4. The Janus particle, as recited in claim 1, wherein said biomolecule-modified region is a continuous region disposed on one side of said low-dimensional substrate.

5. The Janus particle, as recited in claim 1, wherein said biomolecules are uniformly distributed in said biomolecule-modified region.

6. The Janus particle, as recited in claim 1, wherein said biomolecules are antibodies.

7. The Janus particle, as recited in claim 1, wherein said surface of said low-dimensional substrate further comprises a non-biomolecule-modified region, wherein said non-biomolecule-modified region is a continuous region disposed on one side of said low-dimensional substrate.

8. The Janus particle, as recited in claim 7, wherein a surface area of said non-biomolecule-modified region is presented as follows
AM=AS−AB,
wherein
AM represents a total surface area of said non-biomolecule modified region.

9. The Janus particle, as recited in claim 7, further comprising a plurality of magnetic materials disposed in said non-biomolecule-modified region.

10. The Janus particle, as recited in claim 9, wherein said low-dimensional substrate further comprises a plurality of pores, wherein said magnetic materials are disposed in said pores.

11. The Janus particle, as recited in claim 1, wherein said Janus particle further comprises a plurality of fluorescent molecules attached to the surface of said low-dimensional substrate in said biomolecule-modified region.

12. The Janus particle, as recited in claim 9, wherein each of said fluorescent molecules is a FRET donor, a FRET acceptor, or a fluorescent molecule.

13. A tetrahedral structure, comprising:
a microorganism comprising a surface and a plurality of biomolecules disposed on said surface; and
four Janus particles surrounding said microorganism, wherein each of said Janus particles comprises:
a low-dimensional substrate, wherein a surface of said low-dimensional substrate comprises a biomolecule-modified region; and
a plurality of further biomolecules disposed in said biomolecule-modified region, wherein two ends of each of said further biomolecules are respectively attached to said surface of said low-dimensional substrate and each of said biomolecules of said microorganism.

14. The tetrahedral structure, as recited in claim 13, wherein a relationship between a surface area of said biomolecule-modified region and a surface area of said low-dimensional substrate is represented as follows:
(⅕)AS≤AB≤(½)AS,
wherein
AB represents a total surface area of said biomolecule-modified region and
AS represents a total surface area of said low-dimensional substrate.

15. The tetrahedral structure, as recited in claim 13, wherein each of said biomolecules on said surface of said low-dimensional substrate has a specificity to each of said biomolecules on said surface of said microorganism.

16. The tetrahedral structure, as recited in claim 13, wherein each of said Janus particles further comprises a plurality of first fluorescent molecules attached to said surface of said low-dimensional substrate in said biomolecule-modified region, wherein said microorganism further comprises a plurality of second fluorescent molecules attached to said surface of said microorganism, wherein each of said first fluorescent molecules is one of a FRET donor and a FRET acceptor, wherein each of said second fluorescent molecules is another one of said FRET donor and said FRET acceptor.

17. The tetrahedral structure, as recited in claim 13, wherein one of said Janus particles further comprises a plurality of first fluorescent molecules attached to said surface of said low-dimensional substrate in said biomolecule-modified region, wherein another one of said Janus particles further comprises a plurality of second fluorescent molecules attached to said surface of said low-dimensional substrate in said biomolecule-modified region, wherein each of said first fluorescent molecules is one of a FRET donor and a FRET acceptor, wherein each of said second fluorescent molecules is another one of said FRET donor and said FRET acceptor.

18. The tetrahedral structure, as recited in claim 17, wherein said surface of said low-dimensional substrate further comprises a non-biomolecule-modified region, wherein said non-biomolecule-modified region is a continuous region disposed on one side of said low-dimensional substrate.

19. The tetrahedral structure, as recited in claim 18, wherein each of said Janus particles attached with said first fluorescent molecules further comprises a plurality of magnetic materials disposed in said non-biomolecule-modified region of said low-dimensional substrate, wherein each of said Janus particles attached with said second fluorescent molecules comprises no magnetic materials.

20. A method for fabricating a Janus particle, comprising:
(1) providing at least a low-dimensional substrate;
(2) adsorbing said low-dimensional substrate on a surface of a fibrous web structure;
(3) performing a heating process until portions of said low-dimensional substrate are submerged into said fibrous web structure to constitute a submerged portion, wherein other portions of said low-dimensional substrate are exposed from said surface of said fibrous web structure to constitute a protruding portion, wherein a ratio of a surface area of said protruding portion to a surface area of said low-dimensional substrate is between 0.2-0.5;
(4) forming a surface modification layer on a surface of said protruding portion;
(5) detaching said low-dimensional substrate from said surface of said fibrous web structure after the step (4) of forming said surface modification layer; and
(6) disposing a biomolecule layer on said surface modification layer, wherein said biomolecule layer is attached to said surface modification layer.

21. The method for fabricating the Janus particle, as recited in claim 20, wherein said fibrous web structure consists of at least one electrospun fiber, wherein segments of said electrospun fiber are stacked with each other.

22. The method for fabricating the Janus particle, as recited in claim 20, further comprising:
forming a protection layer on said surface modification layer before the step (5) of detaching said low-dimensional substrate from said surface of said fibrous web structure; and
removing said protection layer after the step (5) of detaching said low-dimensional substrate from said surface of said fibrous web structure.

23. The method for fabricating the Janus particle, as recited in claim 20, wherein said low-dimensional substrate is a mesoporous spherical substrate with a plurality of pores.

24. A method of detecting biomolecules, comprising:
(1) providing a plurality of Janus particles, wherein each of the Janus particles comprising:
a low-dimensional substrate, wherein a surface of said low-dimensional substrate comprises a biomolecule-modified region; and
a plurality of biomolecules respectively comprising a fixed end and a free end, wherein each of said fixed ends attached to said surface of said low-dimensional substrate in said biomolecule-modified region, wherein a relationship between a surface area of said biomolecule-modified region and a surface area of said low-dimensional substrate is represented as follows:
(⅕)AS≤AB≤(½)AS,
wherein
AB represents a total surface area of said biomolecule-modified region and
AS represents a total surface area of said low-dimensional substrate;
(2) providing a microorganism, wherein said microorganism comprises a plurality of further biomolecules disposed on a surface of said microorganism; and
(3) mixing said Janus particles and said microorganism so that said free end of each of said biomolecules on said surface of said Janus particles is bound to each of said biomolecules disposed on said surface of said microorganism.

25. The method for detecting the biomolecules, as recited in claim 24, wherein said low-dimensional substrate is a mesoporous low-dimensional substrate.

26. The method for detecting the biomolecules, as recited in claim 24, wherein a relationship of a diameter of said low-dimensional substrate and a diameter of said microorganism is represented as follows:
0.15≤(VD/D)≤0.3,
wherein
D represents said diameter of said low-dimensional substrate and
VD represents said diameter of said microorganism.

27. The method for detecting the biomolecules, as recited in claim 24, wherein said biomolecule-modified region is a continuous region disposed on one side of said low-dimensional substrate.

28. The method for detecting the biomolecules, as recited in claim 24, wherein said biomolecules on said surface of said low-dimensional substrate are uniformly distributed in said biomolecule-modified region.

29. The method for detecting the biomolecules, as recited in claim 24, wherein each of said biomolecules on said surface of said low-dimensional substrate has a specificity to each of said biomolecules on said surface of said microorganism.

30. The method for detecting the biomolecules, as recited in claim 24, wherein each of said Janus particles further comprises a plurality of first fluorescent molecules attached to said surface of said low-dimensional substrate in said biomolecule-modified region, wherein said microorganism further comprises a plurality of second fluorescent molecules attached to said surface of said microorganism, wherein each of said first fluorescent molecules is one of a FRET donor and a FRET acceptor, wherein each of said second fluorescent molecules is another one of said FRET donor and said FRET acceptor.

31. The method for detecting the biomolecules, as recited in claim 24, wherein some of said Janus particles further respectively comprise a plurality of first fluorescent molecules attached to said surface of said low-dimensional substrate in said biomolecule-modified region, wherein others of said Janus particles further respectively comprise a plurality of second fluorescent molecules attached to said surface of said low-dimensional substrate in said biomolecule-modified region, wherein each of said first fluorescent molecules is one of a FRET donor and a FRET acceptor, wherein each of said second fluorescent molecules is another one of said FRET donor and said FRET acceptor.

32. The method for detecting the biomolecules, as recited in claim 31, wherein said surface of said low-dimensional substrate further comprises a non-biomolecule-modified region, wherein said non-biomolecule modified region is a continuous region disposed on one side of said low-dimensional substrate.

33. The method for detecting the biomolecules, as recited in claim 32, wherein each of said Janus particles attached with said first fluorescent molecules further comprises a plurality of magnetic materials disposed in said non-biomolecule-modified region of said low-dimensional substrate, wherein each of said Janus particles attached with said second fluorescent molecules comprises no magnetic materials.

34. The method for detecting the biomolecules, as recited in claim 31, wherein the number of said Janus particles attached with said first fluorescent molecules is equal to the number of said Janus particles attached with said second fluorescent molecules.
